(19) United States
(12) Patent Application Publication
(10) Pub. No.: US 2025/0041042 A1
(43) Pub. Date: Feb. 6, 2025

(54) IMPLANTABLE DEVICE TO FORM A CONSTRICTION WITHIN A BLOOD VESSEL LUMEN

(71) Applicant: Restore Medical Ltd., Or Yehuda (IL)

(72) Inventors: Stephen F. BELLOMO, Zichron Yakov (IL); Ben Adam FRIESEM, Tel Aviv (IL)

(73) Assignee: Restore Medical Ltd., Or Yehuda (IL)

(21) Appl. No.: 18/577,258

(22) PCT Filed: Jul. 6, 2022
(86) PCT No.: PCT/IL2022/050724

§ 371 Date: Jan. 7, 2024

Related U.S. Application Data
(60) Provisional application No. 63/218,966, filed on Jul. 7, 2021.

(51) Int. Cl. A61F 2/07; A61F 2/06

(52) U.S. Cl. CPC A61F 2/07; A61F 2002/068; A61F 2220/0008; A61F 2230/0069; A61F 2230/0078; A61F 2250/0039

(57) ABSTRACT

A device includes a constricting portion and an anchoring element. The anchoring element anchors the device to an inner wall of a blood vessel. The constricting portion extends from an inlet at a proximal end for receiving blood flow therethrough to at least one outlet at a distal end through which flow is expelled. The constricting portion includes a neck section and tapers from the inlet to the neck section. The tapering defines a space substantially surrounding the constricting portion in which flow expelled from the at least one outlet can freely flow.

### RELATED APPLICATION

The present application claims the benefit of priority and under 119 (c) of U.S. provisional application Ser. No. 63/218,966, filed Jul. 7, 2021 by same inventors and with same title, the disclosure of which is incorporated herein by reference.

### FIELD AND BACKGROUND OF THE INVENTION

The present invention, in some embodiments thereof, relates to an implantable device to form a constriction in blood vessel and, more particularly, but not exclusively, to an implantable device configured to improve left ventricle (LV) systolic function in patients with congestive heart failure (CHF) based on forming the constriction in a pulmonary artery.

Congestive heart failure (CHF) is a condition in which the heart does not pump out sufficient blood to meet the body's demands. CHF can result from either a reduced ability of the heart muscle to contract (systolic failure) or from a mechanical problem that limits the ability of the heart's chambers to fill with blood (diastolic failure). When weakened, the heart is unable to keep up with the demands placed upon it and the left ventricle (LV) may get backed up or congested. CHF is a progressive disease. Failure of the left side of the heart (left-heart failure/left-sided failure/left-ventricle failure) is the most common form of the disease.

When CHF due to LV systolic failure occurs, it is typically associated with changes in the geometry of the ventricles, often called remodeling. The LV becomes dilated and the interventricular septum is deflected towards the right ventricle (RV), resulting in decreased LV output/pumping efficiency. The efficient systolic function of the LV is dependent not only on the strength of the myocardium but also on the LV geometry, the position and shape of the interventricular septum and the geometry and function of the RV. Interventricular dependence has been documented in experimental studies which have evaluated both normal and pathological preparations from animals. LV systolic function can be directly influenced by interventions affecting the RV and the position of the interventricular septum.

CHF affects people of all ages including children, but it occurs most frequently in those over age 60, and is a leading cause of hospitalization and death in that age group. Current treatments of CHF include lifestyle changes, medications, and surgical procedures, such as to bypass blocked blood vessels, replace or repair regurgitated or stenotic valves, install stents to open narrowed coronary vessels, install pump assist devices or transplantation of the heart.

U.S. Pat. No. 10,667,931 entitled “Pulmonary artery implant apparatus and methods of use thereof,” the content of which is incorporated by reference herein, discloses an implantable apparatus and methods of use thereof to treat congestive heart failure. The implantable apparatus is anchored by implantation of a section of the apparatus within in a branch pulmonary artery, for example the left pulmonary artery, which then positions and anchors another section, for example a device frame section of the apparatus within the main pulmonary artery. A medical device may be attached to the anchored device frame.

U.S. Patent Publication No. 20180085128, entitled “Artery medical apparatus and methods of use thereof,” the contents of which is incorporated by reference herein, discloses a medical apparatus for deployment within an anatomical blood vessel. The medical apparatus disclosed includes a first tubular wall, a second tubular wall within the first tubular wall, and a constricting element that constricts a circumference of a portion of the second tubular wall. The combination of the first tubular wall, the second tubular wall and the constricting element is disclosed to form a diametrical reducer for reducing an effective diameter of an anatomical blood vessel. It is also disclosed that at least a portion of the second tubular wall is coated with a coating material. Example coating materials disclosed include silicone elastomers, urethane containing polymers, polytetrafluoroethylene, polylactic acid, xenograft or allograft tissue (such as pericardial tissue).

U.S. Patent Publication No. 20180085128, entitled “Double walled fixed length stent like apparatus and methods of used thereof,” the contents of which is incorporated by reference herein, discloses a medical apparatus for deployment within an anatomical blood vessel and methods of use thereof. The medical apparatus includes a first tubular wall and a second tubular wall, placed within the first tubular wall. The first and second tubular walls are firmly connected at their edges and restricted to have same overall longitudinal length. The second tubular wall is configured to be partially constricted towards its inner radial axis, while maintaining its overall longitudinal length. It is also described that the method includes at least partially coating the second tubular wall.

### SUMMARY OF THE INVENTION

According to an aspect of some embodiments there is provided a device configured to form a constriction in a blood vessel and affect a change in pressure upstream and/or downstream the implantable device based on forming the constriction. Optionally and preferably, the change in pressure is selected to improve LV output in patients with CHF and more particularly in patients with a dilated LV. According to some example embodiments, the device is configured to be implanted within a pulmonary artery and is configured to impose a narrowing of the flow path within the pulmonary artery. Optionally and preferably, the imposed narrowing significantly reduces an effective diameter of the pulmonary artery and/or increases flow resistance within the artery and based on this adjusting affects an elevation in RV pressure. The elevated RV pressure may reposition and support an interventricular septum that otherwise may be deflected toward the RV in CHF patients with a dilated LV.

According to some example embodiments, the device is sized and shaped for placement in the branch pulmonary arteries (right and/or left pulmonary arteries). Optionally and preferably, a desired elevation in RV pressure with the device is affected based on implanting a pair of the devices, e.g. one device in each of the right pulmonary artery and the left pulmonary artery. According to some example embodiments, it is desirable to implant the device proximal to a sub-bifurcation of the left or right branch pulmonary arteries to direct all the flow through the device and therefore avoid diversion of flow through a sub-branch artery upstream of the device. In this manner, all flow through the branch pulmonary arteries may be directed through the device rather than be diverted around the device.

There may be a number of potential advantages associated with implanting bilateral devices in the branch pulmonary arteries as opposed to a single device in the main pulmonary artery. One potential advantage is that the relatively longer length of the branch arteries and the smaller diameters as compared to the main pulmonary artery allow for more favorable length to diameter ratio of the device and provides more surface area for securely anchoring the device within the artery. Another potential advantage is that the branch arteries are spaced away from the pulmonary valve. By spacing the device away from the pulmonary valve, the risk of the device moving toward the pulmonary valve and/or interfering with its operation may be significantly reduced.

When implanting a device within the branch pulmonary arteries there is a risk that the device may be overlaid a sub-branch artery and block flow into that sub-branch artery. Since there is significant variability in location of the first sub-branch arteries among individuals, such a risk would be difficult to mitigate simply by selecting a desirable location along the branch artery for implanting and/or based on limiting a length of the device.

According to an aspect of some example embodiments, the device is configured to support backflow along a length of the device to reduce or avoid the risk of blocking sub-branch arteries that may be present along a length of the device. In some example embodiments, a portion of the device is configured to form a seal between with the inner arterial wall to prevent incoming flow from flowing around the device.

According to an aspect of some example embodiments, there is provided a device including a constricting portion extending from an inlet at a proximal end for receiving blood flow therethrough to at least one outlet at a distal end through which flow is expelled; and an anchoring element configured to anchor the device to an inner wall of a blood vessel; wherein the constricting portion includes a neck section and wherein the constricting portion tapers from the inlet to the neck section and wherein the tapering defines a space substantially surrounding the constricting portion in which flow expelled from the at least one outlet can freely flow.

Optionally, the constricting portion includes a flared section that flares from the neck section to the at least one outlet.

Optionally, the anchoring element is an outer frame defining an outer cylindrical wall with openings through which blood may freely flow.

Optionally, the device further includes an inner frame defining an inner cylindrical wall with openings through which blood may freely flow and a cover partially lining the inner frame, wherein the constriction portion is formed by a first portion of the inner frame that is lined with the cover, the first portion extending from the inlet to the at least one outlet.

Optionally, the inner frame is within the outer frame and oriented in a same direction.

Optionally, the inner frame and the outer frame extends from the proximal end to a frame distal end.

Optionally, the inner frame is fixed to the outer frame at each of the proximal end and the frame distal end.

Optionally, a second portion of the inner frame extends beyond the at least one outlet to the frame distal end of the inner frame and wherein flow expelled from the at least one outlet is free to flow through the second portion of the inner frame.

Optionally, the cover lines 50%-90% of a length of the inner frame.

Optionally, the cover extends to the frame distal end and includes openings at the distal end of the constricting portion and wherein the at least one outlet includes the openings formed through the cover at the distal end of the constricting portion.

Optionally, the cover is a liquid impermeable cover.

Optionally, the cover is a pliable material that is fixed on one or both of an inner surface and an outer surface of the inner frame.

Optionally, a diameter of the neck section is 5 mm-15 mm.

Optionally, the anchoring element is fixed to the constricting portion at the proximal end and further includes a circumferential sealing strip extending around the anchoring element at the proximal end.

Optionally, the circumferential sealing strip lines the anchoring element over a length of 3 mm-10 mm from the proximal end.

Optionally, the constricting portion is lined with a cover and wherein the circumferential sealing strip is connected to the cover of the constricting portion with a liquid impermeable sealed connection.

Optionally, the device further includes a constricting element fitted around the neck section and configured to constrict the neck section to a defined diameter.

Optionally, the constricting portion is formed with the neck section in a neutral state absent the constricting element and wherein the constricting element is configured to further constricts the neck section.

Optionally, the constricting element is configured to maintain the defined diameter while implanted within blood vessel.

According to an aspect of some example embodiments, there is provided an outer frame defining an outer cylindrical wall with openings through which blood may freely flow and configured for being anchored within a blood vessel; an inner frame defining an inner cylindrical wall with openings through which blood may freely flow, wherein the inner frame is within the outer frame, and wherein at least a portion of the inner frame is fixedly attached to the outer frame, the inner frame having an inner frame length that extends from a proximal end to a frame distal end; a cover lining the inner frame from the proximal end to a cover distal end to form a constricting portion, wherein the constricting portion has a length that is shorter than the inner frame length, wherein the cover seals the openings in the inner frame over the length of the constricting portion; a constricting element configured to constrict a diameter of the inner frame along the constricting portion to form a neck section with a defined neck diameter, wherein the constricting portion tapers from the proximal end to the neck section and wherein the tapering defines a space substantially surrounding the constricting portion in which flow expelled from constricting portion can freely flow.

Optionally, the cover lines 50%-90% of a length of the inner frame.

Optionally, the cover is a pliable material that is fixed on one or both of an inner surface and an outer surface of the inner frame.

Optionally, a second portion of the inner frame extending from the cover distal end to the frame distal end is not lined with the cover and is exposed.

Optionally, an edge of the inner frame at the cover distal end is configured to be spaced away from an inner wall of a blood vessel when the device is implanted therein.

Optionally, a diameter of the inner frame at the cover distal end is at least 10% smaller than a diameter of the inner frame at the distal end.

Optionally, the outer frame is fixed to the inner frame at the proximal end and further including a circumferential sealing strip extending around the outer frame at the proximal end.

Optionally, the circumferential sealing strip lines the outer frame over a length of 3 mm-10 mm from the proximal end.

Optionally, a diameter of the neck section is 5 mm-15 mm.

Optionally, the neck section is configured to be opened by inserting a balloon through the constricting portion and inflating the balloon therein.

Optionally, the device is collapsible and configured to be implanted within the blood vessel by transcatheter implantation.

Optionally, the spacing is configured to provide free blood flow into branch blood vessels along a length of the blood vessel in which the device is implanted.

Optionally, the device is configured for being implanted within a pulmonary artery.

Optionally, the device is configured for being implanted in the right or left pulmonary artery.

Optionally, the device is configured for elevating pressure in the right ventricle.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are simplified schematic drawings of two example devices in accordance with some example embodiments;

FIGS. 2A and 2B are two example devices with a partially covered inner frame forming a narrowing in accordance with some example embodiments;

FIG. 3 is the example device with an outer circumferential sealing strip in accordance with some example embodiments;

FIG. 4 is the example device with a constricting element, in accordance with some example embodiments;

FIGS. 5A, 5B, 5C and 5D are images of an example device in different configurations, all in accordance with some example embodiments;

FIGS. 6A and 6B are two drawings of an example inner frame of the device shown without and with its cover, both in accordance with some example embodiments;

FIG. 7 is a drawing of an example anchoring element without a circumferential sealing strip, in accordance with some example embodiments;

FIGS. 8A and 8B are two images of a flaring section of an example device from the distal end (flow discharge), shown in an open and constricted configuration respectively, both in accordance with some example embodiments; and

FIG. 9 is a schematic drawings of example devices implanted in each of the left and right pulmonary artery in accordance with some example embodiments.

### DESCRIPTION OF SPECIFIC EMBODIMENTS OF THE INVENTION

The present invention, in some embodiments thereof, relates to an implantable device to form a constriction in a blood vessel and, more particularly, but not exclusively, to an implantable device configured to improve left ventricle (LV) systolic function in patients with congestive heart failure (CHF) based on forming the constriction in a pulmonary artery.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

According to some example embodiments, the implantable device includes both a constricting portion, e.g., a diameter constricting portion, to direct the flow in the blood vessel through a defined narrowing and an anchoring element to anchor the constricting portion to an inner wall of the blood vessel. The constricting portion may extend between a first opening (an inlet) at a proximal end through which flow in blood vessel is received and at least one second opening (an outlet) at a distal end through which flow is expelled from the device. According to some example embodiments, the inlet has a diameter that is substantially the same as a diameter of the blood vessel when implanted therein and the constricting portion narrows with respect to the inlet. According to some example embodiments, the diameter of both an inner surface and the outer surface of the constricting portion is narrowed in relation to the inlet. The outer surface as defined herein refers to the abluminal surface of the constricting portion that faces the inner wall of the blood vessel (away from the lumen) when implanted therein. The inner surface as defined herein refers to the luminal surface of the constricting portion that faces the lumen within the constricting portion through which the flow in the blood vessel is directed. The shape and extent of the narrowing of the outer surface may be the same or different than that of the inner surface.

According to some example embodiments, the narrowed diameter of the outer surface is configured for defining a volume around the outer surface through which blood may flow to feed any sub-branch blood vessels positioned along a length of the constricting portion and the at least one second opening includes an opening that provides flow access (a flow path) to the volume around the outer surface.

Optionally, the at least one second opening is a single opening with an outlet diameter that is smaller than a diameter at the inlet. In some example embodiments, the smaller opening at the distal end is configured to be at least partially spaced away from an inner blood vessel wall when the device is implanted therein. Optionally, the second opening is at least partially spaced away from the anchoring element. Spacing between the second opening and the anchoring element provides a backflow path along a length of the device.

Optionally and preferably, the constricting portion tapers from its proximal end towards a neck section and then flares from the neck section to its distal end. Optionally, the neck section is a radial neck section. Optionally, the neck section is off-centered over a length of the device and closer to the distal end than to the proximal end. Optionally, the neck section is at a position corresponding to 50%-90% of the device length from the proximal end. Alternatively, the neck section may be centered over a length of the device. The flaring angle between the neck section and the distal end may or may not correspond to the tapering angle between the proximal end to the neck section. Optionally, a diameter of the constricting portion at the distal end is 2 mm-20 mm less than the diameter of the constricting portion at the proximal end in an implanted state (or in its fully expanded state). Optionally, an inlet diameter of the constriction portion at the proximal end is 25 mm-35 mm, e.g., 30 mm. Alternatively, constricting portion does not include the flaring section between the neck section and the distal end. According to some example embodiments, the proximal end of the constricting portion is configured to abut the inner blood vessel wall and direct the flow therethrough while the distal end is configured to be at least partially spaced away from the inner arterial wall to allow backflow between the constricting portion and the inner wall along a length of the constricting portion.

In some example embodiments, the constricting portion is a cylindrical body including the neck section. Optionally and preferably, the neck section has a diameter that is 5 mm-15 mm. According to some example embodiments, a diameter of the narrowing, e.g. the neck section is configured to be substantially maintained throughout the cardiac cycle and over the period of treatment. Optionally, the narrowing is released (or opened) manually during a catheterization procedure. In some example embodiments, the narrowing is released and/or expanded with a balloon inserted and inflated therein. Release of the narrowing may be actuated to terminate the treatment. Optionally, the device accommodates some variation in neck diameter due to pulsating flow, e.g. 0%-20%. Optionally, and preferably, a selected diameter for the narrowing is supported with a dedicated constricting element that is maintained on the cylindrical body. Optionally, the constricting element is in the form of a ring-shaped element and/or a wire wrapped and secured around the cylindrical body. According to some example embodiments, the constricting element is configured to support the narrowing at the neck section over an extended period of time, e.g. over a plurality of years.

According to some example embodiments, the cylindrical body is a collapsible frame that is configured to expand to a defined shape. Optionally and preferably, the frame is formed from shape memory material. According to some example embodiments, the frame is at least partially lined and/or encapsulated with a cover and the frame together with the cover define the constricting portion of the device. Optionally and preferably, the cover is a liquid impermeable lining (or membrane). Optionally, the cover is a pliable material that may form folds and/or creases when further constricting the neck section with the constricting element. Example materials for the cover include PTFE, polyurethane and silicone. In some example embodiments, the cover may have elastic properties. Optionally, the clastic properties provide reducing or avoiding folds over a range of diameters for the neck section when further constricting the neck section with the constricting element. Optionally, the cover is formed with a polymer material.

As defined herein, the portion of the frame that is lined with the cover defines the constricting portion of the frame that extends between the proximal end and the distal end. Optionally, the cover is applied to the frame while the frame is in its neutral configuration, e.g. fully expanded. In some example embodiments, the neutral configuration includes a neck section, which may be significantly larger in diameter than an operational constriction diameter used for treatment. Optionally, in operation, the neck section is constricted to a smaller diameter (the operational diameter) with the constricting element. The constricting element may be positioned before or after implantation.

According to some example embodiments, the constricting portion is formed with a frame that is partially covered with the cover. Optionally and preferably, the cover is not along the entire length of the frame so that a portion of the frame extending past the distal end of constricting portion is exposed and allows flow therethrough. In some example embodiments, the exposed portion of the frame is configured to flare to a substantially same diameter as the proximal end of the constricting portion in its neutral or fully expanded state. Optionally, the frame is fixed to the anchoring element at one or both ends.

According to some example embodiments, the anchoring element is an outer frame (or second frame) that is configured to substantially surround the constricting portion. According to some example embodiments, the anchoring element is fixedly attached to the proximal end of the constricting portion, the distal end, both the proximal end and the distal end, or anywhere else along the constricting portion. Optionally, when the constricting portion is formed with a frame that is partially lined with a cover, the anchoring element is fixedly attached to one or both ends of the frame. In some example embodiments, the anchoring element is configured to promote tissue ingrowth and/or provide an atraumatic interface while anchoring the device within the inner wall of the blood vessel. Optionally, the outer frame and the inner frame are defined to have the same length. In other example embodiments, the outer frame may be either longer or shorter than the inner frame. Optionally, more than one anchoring element may be used to anchor the device. Optionally, a first anchoring element may be configured to anchor a proximal end of the constricting portion and a second anchoring element may be configured to anchor a distal end of the constricting portion within the arterial inner wall. Optionally, each of the first and second anchoring elements is a collapsible frame.

According to some example embodiments, the device is further fitted with a circumferential sealing strip extending from the proximal end and partially covering, e.g. lining the anchoring element. In some example embodiments, the circumferential sealing strip extends over a length of 2 mm-10 mm or 3 mm-8 mm, e.g. 5 mm or 7.5 mm. In some example embodiments, when the anchoring element is a collapsible frame, the circumferential scaling strip is configured to extend to a first array of strut junctions on the collapsible frame. According to some example embodiments, the circumferential sealing strip is configured to enhance a seal between the proximal end of the constricting portion and the inner wall of the blood vessel. According to some example embodiments, the length of the circumferential sealing strip is selected to provide an adequate seal while reducing a risk of covering sub-branch blood vessels, e.g. sub-branch pulmonary arteries. In some example embodiments, the circumferential scaling strip is configured to be fixed to and/or integrated with the proximal end of the cover lining the inner frame. Optionally and preferably, the circumferential sealing strip is formed with pliable material, e.g. with the same material used for the cover lining of the inner frame, e.g. PTFE, polyurethane or silicone. Optionally, when the device includes more than one anchoring elements, the circumferential sealing strip is configured to line the anchoring element at the proximal end.

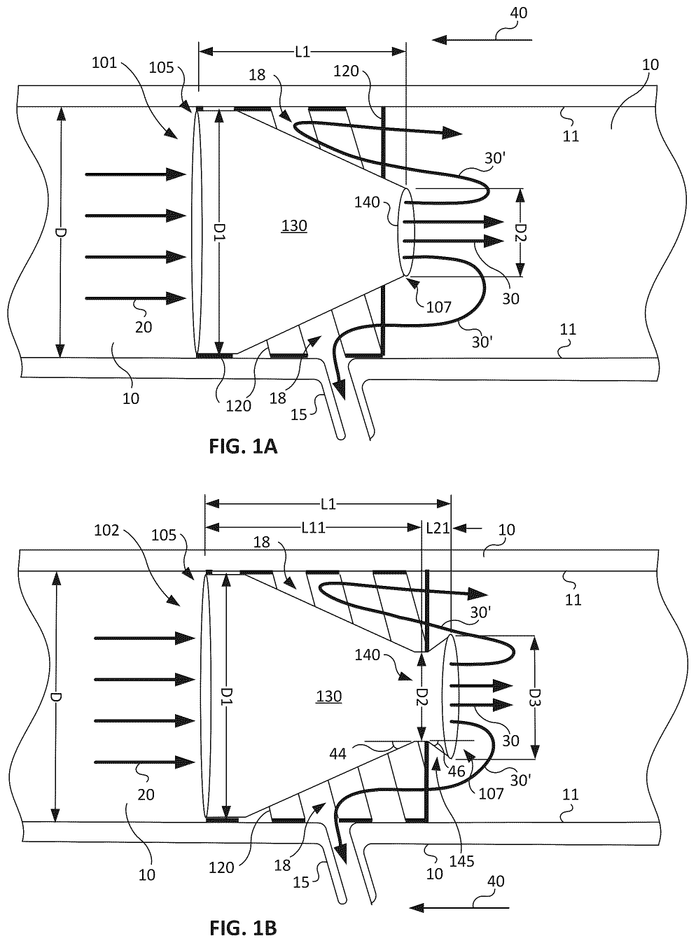

Reference is now made to FIGS. 1A and 1B showing simplified schematic drawings of two example devices in accordance with some example embodiments. According to some example embodiments, a device, e.g. device 101 or device 102 include a constricting portion 130 though which flow 20 in an artery or other blood vessel 10 is directed and an anchoring element 120. Anchoring element 120 is configured to prevent shifting of constricting portion 130 along a length of blood vessel 10 and optionally and preferably to support a defined orientation of constricting portion 130 within blood vessel 10 when implanted therein. According to some example embodiments, anchoring element 120 is a cylindrical structure with substantially constant diameter that corresponds to diameter D of blood vessel 10. In some example embodiments, anchoring element 120 is a frame, e.g. a mesh and/or a stent that is configured to engage and/or abut inner wall 11 of blood vessel 10 without blocking flow into a sub-branch artery 15 along a length of anchoring element 120. Optionally and preferably, anchoring element 120 is collapsible. Optionally and preferably, anchoring element 120 is configured to self-expand from a collapsed state to an operational state. Optionally and preferably, in an operational state, anchoring element 120 assumes a diameter D of blood vessel 10.

According to some example embodiments, constricting portion 130 is shaped to taper from a proximal end 105 to a neck section 140 having a diameter D2. Optionally and preferably, constricting portion 130 is fixed to anchoring element 120 at proximal end 105 and a diameter D1 of constricting portion 130 at proximal end 105 may be substantially the same as diameter D of blood vessel 10 when implanted therein. According to some example embodiments, tapering of constricting portion 130 defines a space and/or volume 18 surrounding constricting portion 130 in which blood flow 30′ exiting constricting portion 130 may flow in an upstream direction 40 along a length L1 to feed sub-branch artery 15.

According to some example embodiments, distal end 107 of constricting portion 130 is spaced away from anchoring element 120 and/or inner wall 11 to create flow access into volume 18 between constricting portion 130 and inner wall 11. According to some example embodiments, blood flow 30 exiting flow constricting device 120 is free to flow downstream and also in an upstream direction 40, e.g., flow 30′ over a length L1 of constricting portion 130. Flow 30 may typically exit the device (e.g., device 101 or device 102) with some turbulence so that while some of flow 30 flows in a natural downstream direction through blood vessel 10, a portion of the flow (flow 30′) will flow in space 18 between constricting portion 130 and inner wall 11 of blood vessel 10 creating backflow 30′. Optionally and preferably, the backflow 30′ of blood in an upstream direction 40 (backflow) is blocked at proximal end 105. Flow into volume 18 may provide feeding any sub-branch artery (or arteries) 15. Any excess backflow 30′ that is not fed into sub-branch arteries 15 may reverse direction and flow downstream blood vessel 10.

Optionally, anchoring element 120 includes one or more structures to support constricting portion 130, neck section 140 and/or flared section 145 in a concentric orientation with respect to anchoring element 120 and/or blood vessel 10. In other example embodiments, neck section 140 may be configured to be non-concentrically oriented with respect to anchoring element 120 and/or blood vessel 10, and anchoring element 120 supports the tapered structure and/or neck section 140 in the defined non-concentrically orientation.

According to some example embodiments, the device (device 101 or device 102) is implanted in a pulmonary artery, e.g. a branch pulmonary artery and diameter D2 is defined to affect a desired raise in pressure in the right ventricle of the heart upstream from the device (device 101 or device 102).

In some example embodiments as shown in FIG. 1A, an example device 101 including neck section 140 is at a distal end 107 of constricting portion 130 and flow 30 exiting constricting portion 130 exits from neck section 140. Neck section 140 is defined to have a diameter D2 that is smaller than diameter D1 at proximal end 105. In some example embodiments, D2 is 5 mm-15 mm or 7 mm-10 mm. According to some example embodiments, neck section 140 is configured to be at least partially spaced away from inner wall 11 of blood vessel 10 when implanted therein. According to some example embodiments, neck section 140 provides flow access to volume 18 that may otherwise have been blocked based on diverting flow in the artery through constricting portion 130. According to some example embodiments, based on the defined geometry of constricting portion 130, flow exiting neck section 140 is free to flow upstream (flow 30′) into volume 18 as well as downstream (flow 30) in its natural direction through blood vessel 10.

In some example embodiments, length L1 is 0 mm-40 mm and D1 is 25 mm-35 mm in a neutral state (prior to being implanted within blood vessel 10). Optionally and preferably, the device is in a partially compressed state when implanted within blood vessel 10 so that D1 while implanted within blood vessel 10 may be less than D1 prior to being implanted.

In some example embodiments, as shown in FIG. 1B, an example device 102 includes a flared section 145 in constricting portion 130 that extends downstream from neck section 140 to distal end 107. According to some example embodiments, flared section 145 at distal end 107 has a diameter D3 that is larger than diameter D2 of neck section 140 and smaller than diameter D1 of constricting portion 130 at proximal end 105 while implanted within artery 10. According to some example embodiments, D3 is defined to be at least 10% smaller than D1. According to some example embodiments, D3 is at least 10% smaller than D1 at its neutral state (prior to being implanted within blood vessel 10). Optionally, D3 is 15 mm-27 mm and D1 is 28 mm-32 mm at its neutral state (prior to being implanted within blood vessel 10).

According to some example embodiments, flared section 145 is configured to be at least partially spaced away from inner wall 11 of blood vessel 10 when implanted therein to provide flow access to volume 18 that may otherwise have been blocked based on diverting flow in the artery through constricting portion 130. According to some example embodiments, flow exiting flared section 145 is free to flow upstream (flow 30′) into volume 18 as well as downstream (flow 30) in its natural direction through blood vessel 10.

In some example embodiments, flared section 145 may flare at an angle 46 that is wider than an angle 44 of tapering. In other example embodiments, flaring angle 46 and tapering angle 44 may be the same or flaring angle 46 may be less than tapering angle 44.

According to some example embodiments when constricting portion 130 includes flared section 145, neck section 140 is positioned around a middle of constricting portion 130 or toward a distal end 107. For example, L11 may be 40%-90% of L1.

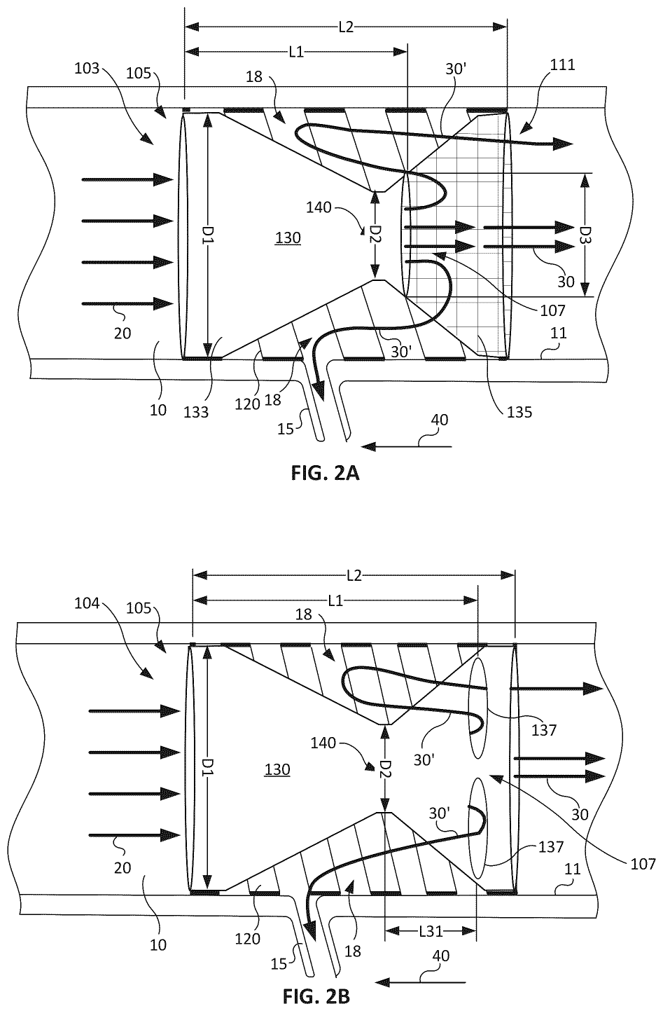

Reference is now made to FIGS. 2A and 2B showing two example devices with a partially covered inner frame forming a narrowing in accordance with some example embodiments. According to some example embodiments, a device, e.g. device 103 or device 104 includes anchoring element 120 and an inner frame 135 that is partially lined with a cover 133. Optionally and preferably, inner frame 135 is a mesh and/or a stent that is collapsible. Optionally and preferably, inner frame 135 is configured to self-expand from a collapsed state to an operational state. In some example embodiments inner frame 135 is formed with a shape memory material and/or shape memory alloy, e.g., Nitinol. Inner frame 135 may be braided or may be tube that is cut with a meshed pattern.

According to some example embodiments, cover 133 extends from proximal end 105 to distal end 107. Optionally and preferably, cover 133 is a liquid impermeable cover or lining. According to some example embodiments, the partial lining of inner frame 135 with cover 133 forms constricting portion 130. Optionally and preferably, inner frame 135 includes neck section 140. Constricting portion 130 is defined herein as a portion of inner frame 135 that is lined with cover 133 and extends from proximal end 105 to distal end 107. Distal end 107 may be at neck section 140 (similar to device 101 in FIG. 1A) or at a defined distance downstream neck section 140 to define a flared section 145. Flared section 145 may be as described in FIG. 1B. According to some example embodiments, distal end 107 of constricting portion 130 includes an opening through which flow within constricting portion 130 may be expelled.

Optionally and preferably, both anchoring element 120 and inner frame 135 are collapsible frames. Inner frame 135 may be positioned within anchoring element 120 and may be aligned in a same direction as anchoring element 120. According to some example embodiments, inner frame 135 is fixedly attached at least at proximal end 105 to anchoring element 120. In some example embodiments, inner frame 135 and anchoring element 120 have a same length L2. In some example embodiments, inner frame 135 is fixedly attached at both ends to anchoring element 120, e.g., at proximal end 105 and at frame distal end 111. Optionally, inner frame 135 is fixed to anchoring element 120 at least at each of proximal end 105 and at frame distal end 111.

According to some example embodiments, cover 133 is formed from a pliable material. Optionally, cover 133 is PTFE, polyurethane, silicone or a hybrid material, e.g. an electrospun polytetrafluoroethylene/polyurethane (PTFE/PU) composite. Cover 133 may be sewn, welded, glued or otherwise attached to inner frame 135. In some example embodiments, cover 133 includes an inner cover that lines an inner surface of a portion of inner frame 135. Optionally, cover 133 includes an outer cover that lines an outer surface of a portion of inner frame 135. Optionally and preferably, cover 133 includes both an inner cover and an outer cover, encapsulating inner frame 135. Optionally, inner cover and outer cover may be sewn, welded, glued or otherwise connected to form cover 133.

According to some example embodiments, the device (device 103 or device 104) is implanted in a pulmonary artery, e.g. a branch pulmonary artery, and diameter D2 is defined to affect a desired raise in pressure in the right ventricle of the heart upstream of the device.

Referring now to FIG. 2A, in some example embodiments, impermeable cover 133 extends past neck section 140 to form a flared section 145 through which flow 30 exits from constricting portion 130. According to some example embodiments, flow 30 exiting constricting portion 130 is free to flow in and out of volume 18 between constricting portion 130 and inner wall 11, e.g. flow 30′. D1, D2, D3 and L1 may be as described herein, e.g. in reference to FIGS. 1A and 1B. In some example embodiments, a diameter of inner frame 135 at frame distal end 111 may be substantially the same as D1 in a neutral state of device 103 (prior to implanting). In some example embodiments, L2 is 28 mm-32 mm, e.g. 30 mm. Optionally, neck section 140 is positioned anywhere between substantially in a center of inner frame 135 and 1-5 mm from distal end 107.

Referring now to FIG. 2B, in some example embodiments, impermeable cover 133 extends along an entire length L2 of device 104 with one or more openings 137 through which backflow 30′ may flow toward sub-branch artery 15. Optionally and preferably, one or more openings 137 are configured to provide free flow into and out of space 18. Constricting portion 130 in this embodiment is defined as a portion of inner frame 135 that extends from proximal end 105 to one or more openings 137. D1, D2, D3, L1 and L2 may be as described herein, e.g. in reference to FIGS. 1A, 1B and 2A. Length L31 may be 1 mm-4 mm and/or L31 is 10% of L2.

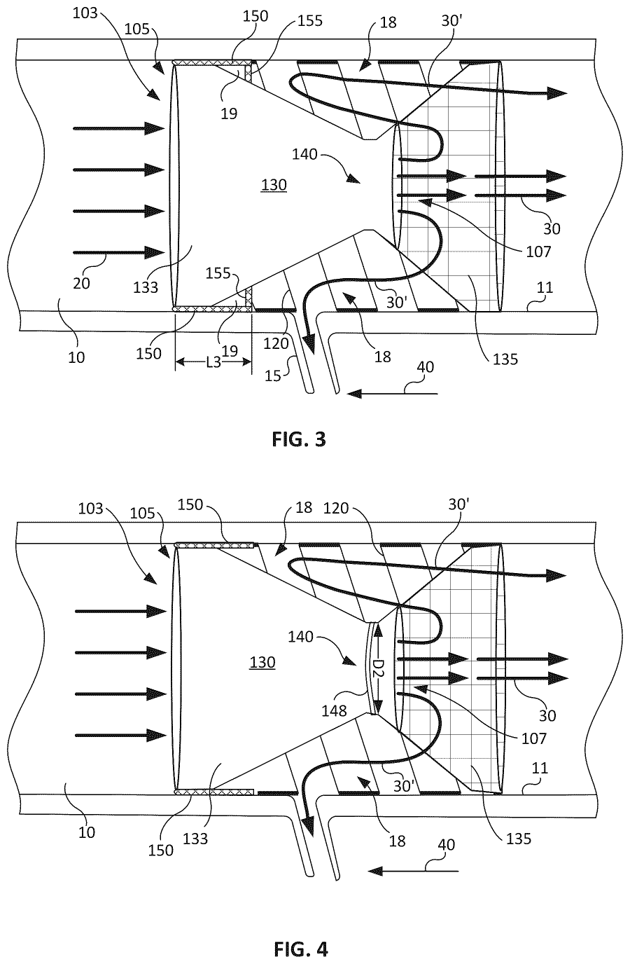

Reference is now made to FIG. 3 showing the example device with an outer circumferential sealing strip in accordance with some example embodiments. According to some example embodiments, device 103 includes a circumferential scaling strip 150 that lines a portion of anchoring element 120. Circumferential sealing strip 150 may be sewn, welded, glued or otherwise attached to anchoring element 120. Optionally, circumferential sealing strip 150 is an integral part of anchoring element 120, e.g. a liquid impermeable portion of anchoring element 120. In some example embodiments, circumferential sealing strip 150 includes an inner cover that lines an inner surface of anchoring element 120, an outer cover that lines an outer surface of anchoring element 120 or both an inner cover and an outer cover.

According to some example embodiments, circumferential scaling strip 150 is configured for improving a sealed engagement of proximal end 105 of constricting portion 130 with inner wall 11 so that flow 20 within blood vessel 10 will be directed through constricting portion 130 and not leak around constricting portion 130. According to some example embodiments, circumferential sealing strip 150 further provides a seal configured to prevent backflow 30′ from flowing upstream proximal end 105.

In some example embodiments, it is advantageous to limit a length of circumferential scaling strip 150 to prevent a risk that circumferential sealing strip 150 may cover, e.g. block entrance into sub-branch artery 15 extending from blood vessel 10 in which device 103 is implanted while being long enough to provide the desired sealing. In some example embodiments, circumferential sealing strip 150 is defined to have a length L3 of 2 mm-10 mm or 3 mm-8 mm, e.g., 5 mm or 7.5 mm.

In some example embodiments, sealing strip 150 includes or is integrally connected to an annular extension 155 configured to block flow 30′ from entering a pocket space 19 between circumferential sealing strip 150 and cover 133.

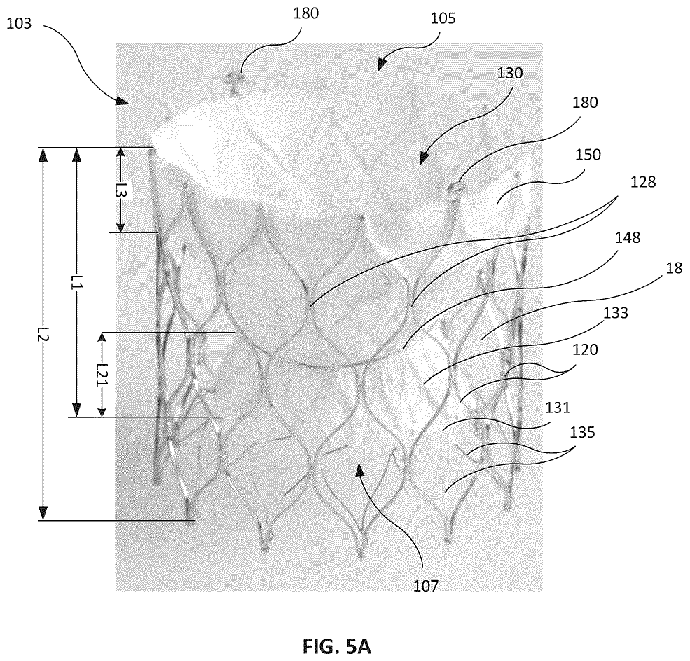

Reference is now made to FIG. 4 showing the example device with a constricting element, in accordance with some example embodiments. According to some example embodiments, a diameter of neck section 140 is set with a constricting element 148 that encompasses and/or is wrapped around constricting portion 130. In some example embodiments, constricting element 148 is configured to narrow neck section 140 to obtain a desired narrowing, e.g., a desired diameter D2. In some example embodiments, constricting element 148 is configured to support substantially maintaining the narrowing over pulsating flow through the artery. Optionally, a 0%-20% change in diameter may be expected over the cardiac cycle with constricting element 148. Constricting element 148 may be a Nitinol wire or tube. Optionally, a diameter D2 may be defined by fixing ends of constricting element 148, e.g. by welding, clasping or fastening. In other example embodiments, constricting element 148 may be ring piece or a clasp. Optionally, constricting portion 130 is pre-formed with neck section 140 and constricting element is configured to further constrict neck section 140. In these example embodiments, D2 without constricting element 148 is 15 mm-30 mm and with constricting element 148 is 5 mm-15 mm. In other example embodiments, is not pre-formed with neck section 140. Optionally, a width or shape of constricting element 148 may be selected to provide a desired geometry for neck section 140. In some example embodiments, the further constriction of neck section 140 with constricting element 148 introduces ruffles, creases or folds in impermeable cover 133. According to some example embodiments, the constricting element 148 is adjustable and/or openable. Adjusting or opening constricting element 148 may optionally provide terminating the treatment provided by the device without the need to remove the device from the artery.

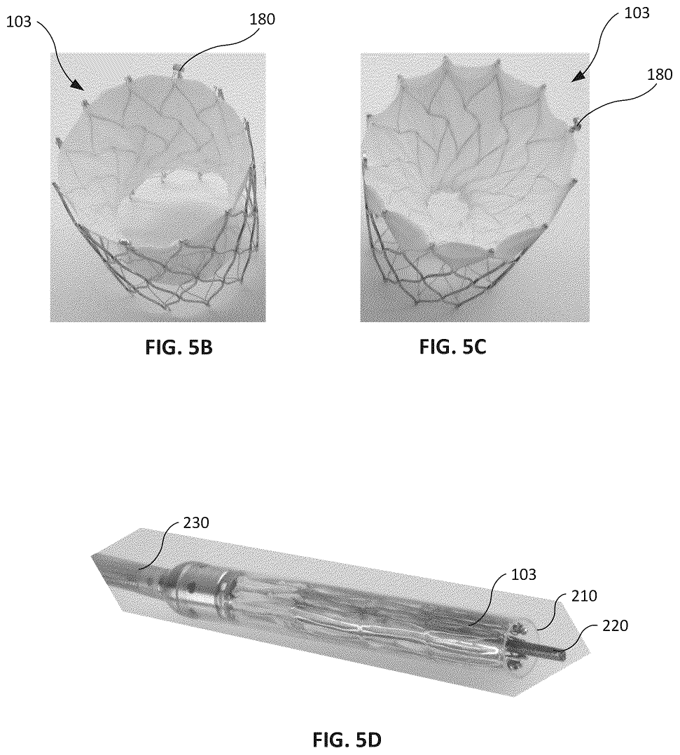

Reference is now made to FIGS. 5A, 5B, 5C and 5D showing images of an example device in different configurations, all in accordance with some example embodiments. According to some example embodiments, flow through an artery may be directed into constricting portion 130 from proximal end 105 and exit constricting portion 130 at distal end 107. Optionally, flow exiting distal end 107 may continue to flow downstream or may flow around an edge 131, e.g., a flare of constricting portion 130 at distal end 107 and then flow through space 18 between constricting portion 130 and anchoring element 120 in a direction upstream toward proximal end 105.

In some example embodiments, circumferential sealing strip 150 is defined to extend to a first array of strut junctions 128 formed in anchoring element 120. In some example embodiments, favorable scaling is obtained based on extending circumferential sealing strip 150 to first array of strut junctions 128. According to some example embodiments, circumferential scaling strip 150 and cover 133 are integral or fixedly connected with a liquid impermeable seal at proximal end 105. Optionally, circumferential scaling strip 150 is an extension of cover 133 that is folded over anchoring element 120.

According to some example embodiments, device 103 is configured to be implanted in a blood vessel by a trans-catheterization procedure. Optionally, device 103 includes one or more holding elements 180 with which device 103 may be manipulated during the catheterization procedure. Optionally, holding element 180 are formed on anchoring element 120. Optionally, holding element 180 are positioned near or at proximal end 105.

FIG. 5B is an example image of device 103 in a fully opened state, e.g. maximum radial neck section. Device 103 is an example device that is pre-formed with neck section 140. FIG. 5C is an example image of device 103 in a further constricted state in which the constriction element 148 is tightened around the radial neck section. FIG. 5D is an example image of device 103 in a delivery system. During delivery, e.g., implantation, device 103 may be collapsed within delivery capsule 210. Optionally, device 103 may be collapsed around a guide wire lumen 220 extending through a delivery system sheath 230.

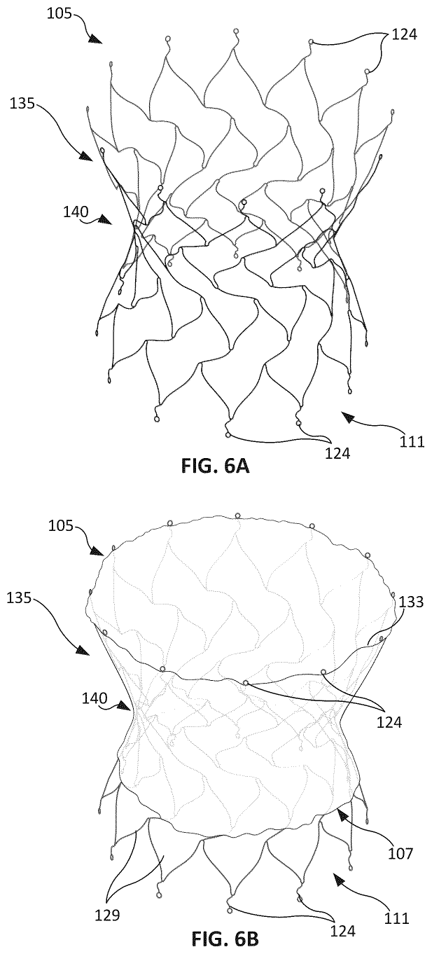

Reference is now made to FIGS. 6A and 6B showing two drawings of an example inner frame including the constricting portion 130 shown without and with its cover, both in accordance with some example embodiments. According to some example embodiments, inner frame 135 is a collapsible frame formed from a shape memory material. Optionally and preferably, inner frame 135 includes neck section 140 with a slight narrowing in its neutral state. According to some example embodiments, neck section 140 may be adjusted, e.g., further constricted with a constricting element that engages neck section 140. Optionally and preferably, neck section 140 in its neutral state is not configured to affect any substantial pressure differential when implanted within a blood vessel. Optionally and preferably, a desired pressure elevation is provided based on further constricting neck section 140 with the constricting element. In some example embodiments, the slight narrowing in the neutral state predisposes inner frame 135 to a desired shape, reduces fatigue of material at neck section 140 that may occur when further constricting with the constricting element and also provides reducing the amount of ruffling of material 133 that occurs when further constricting with the constricting element.

According to some example embodiments, inner frame 135 includes an array of weld eyelets 124 that define each of a proximal end 105 and a frame distal end 111. Optionally, one or more of weld eyelets 124 are welded or otherwise connected to corresponding weld eyelets on anchoring element 120 to form device 103.

According to some example embodiments, inner frame 135 is partially lined (or covered) with a cover 133 that is liquid impermeable. According to some example embodiments, cover 133 extends from one end of inner frame 135, e.g. proximal end 105 past neck section 140 to a distal end 107 that is spaced away from frame distal end 111. According to some example embodiments, flow entering through the portion of inner frame 135 lined with cover 133 at proximal end 105 and out from distal end 107 is free to flow across struts 129 of a portion of inner frame 135 extending between distal end 107 and frame distal end 111.

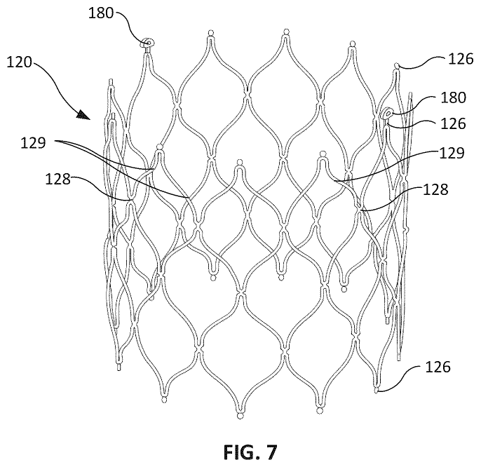

Reference is now made to FIG. 7 showing a drawing of an example anchoring element shown without a circumferential scaling strip, in accordance with some example embodiments. Optionally and preferably, the anchoring element 120 is cylindrical in shape and has a constant diameter along its length in its neutral state, e.g. its fully expanded state. Optionally, anchoring element 120 may vary somewhat in diameter while implanted within a blood vessel based on the mechanical properties of the blood vessel. According to some example embodiments, anchoring element 120 is a frame, e.g., a mesh including strut elements 129 joined at strut junctions 128.

In some example embodiments, one and/or both ends of anchoring element 120 is defined by an array of weld eyelets 126. Optionally, one or more of these weld eyelets 126 are configured to be welded or otherwise connected to corresponding weld eyelets 124 on inner frame 135 to form device 103. Optionally, one or more of weld eyelets 126 includes holding element 180.

According to some example embodiments, openings between strut junctions 129 allows substantially free blood flow across the frame body. Optionally and preferably, anchoring element 120 is configured to be collapsible. Optionally and preferably, anchoring element 120 is made from shape memory material. Optionally, anchoring element 120 is formed from Nitinol or other shape memory alloy and/or metal material suitable for implanting in blood vessel. Optionally, anchoring element 120 may be a mesh formed by cutting a tube or by braiding a wire. Optionally, anchoring element 120 is configured to be self-expanding. Optionally, anchoring element 120 is configured to be balloon expandable.

Optionally and preferably, circumferential sealing strip 150 lines a portion of anchoring element 120 that extends from one end of anchoring element 120, preferably from the proximal end. In some example embodiments, circumferential sealing strip 150 is configured to extend from weld eyelets 126 and to at least to a first array of strut junctions 128. Optionally, one or more of holding elements 180 may be exposed, e.g. may not be lined with circumferential scaling strip 150. In some example embodiments, circumferential sealing strip 150 extends over a length L3 of 2 mm-10 mm or 3 mm-8 mm, e.g., 5 mm or 7.5 mm. In some example embodiments, extending circumferential sealing strip 150 up to first strut junctions 128 improves the scaling effect of circumferential scaling strip 150 and avoids leakage through a flap for fold that may occur in circumferential sealing strip material when not fully stretched between weld eyelets 128.

In some example embodiments, inner frame 135 including the constricting portion 130 is positioned within anchoring element 120 and fixed on anchoring element 120. Optionally, the fixing includes connecting one or more eyelets 124 to corresponding eyelets 126. In some example embodiments, eyelets 124 are welded to eyelets 126. Inner frame 135 may be fixed to anchoring element 120 at proximal end 105, at frame distal end 111, or both proximal end 105 and frame distal end 111.

Referring back to FIG. 5A, according to some example embodiments, circumferential sealing strip 150 on anchoring element 120 is connected or fixed to cover 133 with a seamless connection. Optionally and preferably, circumferential scaling strip 150 is lined over an outer surface of anchoring element 120 after fixedly attaching inner frame 135 to anchoring element 120. Circumferential scaling strip 150 and covering 133 may be connected to each other or may be integral, e.g., optionally cover 133 may fold over the proximal edge 105 and cover the outer surface of the anchoring element 120.

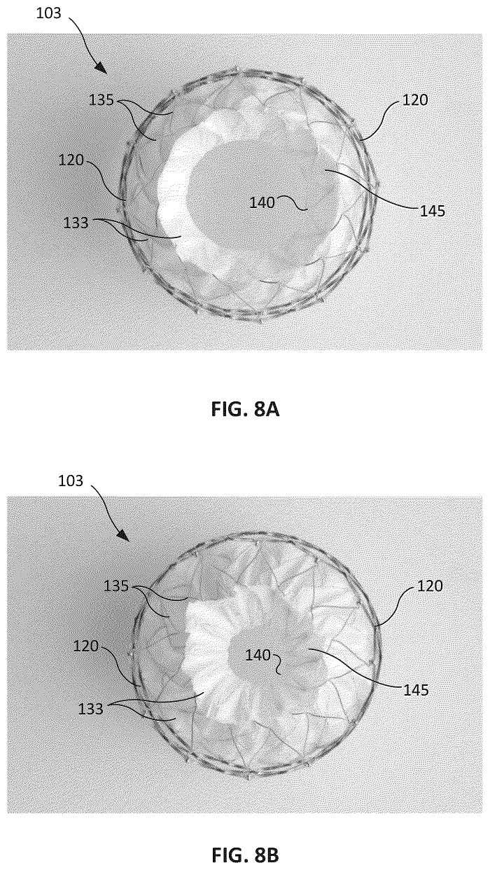

Reference is now made to FIGS. 8A and 8B showing two images of a flaring section of an example device from the distal end (flow discharge), shown in an open and constricted configuration respectively, both in accordance with some example embodiments. According to some example embodiments, cover 133 partially covers inner frame 135 to form a flow constriction section that tapers to a neck section 140 and then flares along a flaring section, e.g. flared section 145 (FIG. 1B). In some example embodiments, neck section 140 is adjustable. Optionally, a diameter of neck section 140 is defined with a constricting element 148 (FIG. 4) that is positioned around neck section 140.

In some example embodiments, cover 133 is formed from a pliable material. Optionally in an open configuration of flow constricting element 130, cover 133 lines inner frame 135 with no creases or folds (FIG. 8A). Optionally, when constricting neck section 140 is constricted by constricting element 148, creases, ruffles and/or folds may be formed with the cover material of cover 133 (FIG. 8B). In other example embodiments, ruffling is reduced and/or avoided by using a material with enhanced clastic or elastomeric material for cover 133.

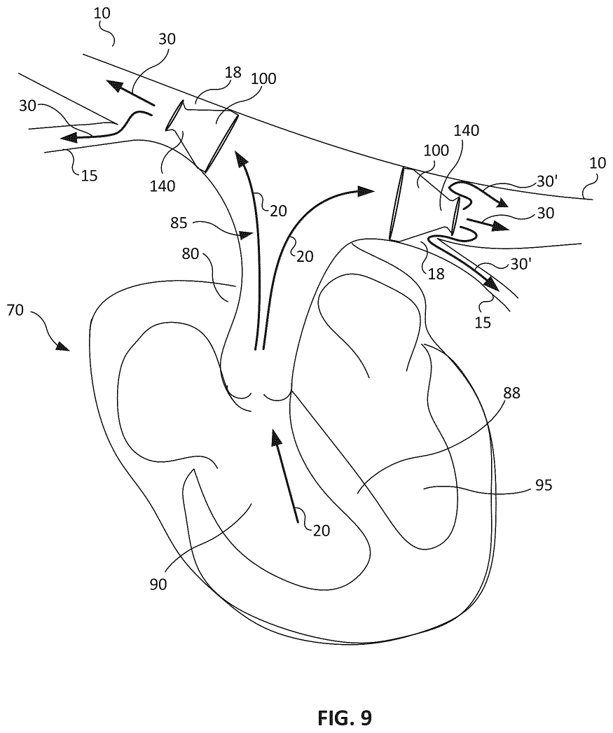

Reference is now made to FIG. 9 showing a schematic drawings of example devices implanted in each of the left and right branch pulmonary arteries in accordance with some example embodiments. According to some example embodiments, a device 100 is implanted in one or more of branch pulmonary arteries 10, e.g., left pulmonary artery and right pulmonary artery. Device 100 generally represents a device configured to form a constriction for flow through blood vessel 10 and may be any one of devices 101, 102, 103 and 104. Blood flow 20 from right ventricle 90 of heart 70 flows into main pulmonary artery 80 and to each of branch arteries 10. According to some example embodiments, neck section 140 constricts flow 20 and creates back pressure in right ventricle 90. Optionally, the back pressure is selected to affect a shift in septum 88 toward left ventricle 95.

According to some example embodiments, one or more of devices 100 are implanted near a bifurcation 85 of main pulmonary artery 80. Optionally, implanting device 100 near bifurcation 85 avoids a risk of diverting flow 20 into a sub-branch artery 15 upstream from device 100. Such a diversion may potentially lead to excessive flow through such a sub-branch artery 15 and limit the ability to increase back pressure in the right ventricle 90. This excessive flow may be undesirable as it may overload on the sub-branch artery and lead to an uneven distribution of flow in the lung, e.g., one lobe of the lung will receive excessive blood flow and proportionally decrease flow to the other lobes.

According to some example embodiments, device 100 is configured to allow back flow 30′ around device 100 so that any sub-branch artery 15 that is covered by device 100 may have access to flow 20 from main pulmonary artery 80. Neck section 140 may have a same or different diameter D2 in each of branch arteries 10. Optionally, a diameter D2 of neck section 140 in each of devices 100 is defined to maintain a desired split of flow 20 from main pulmonary artery 80 into left branch artery and right branch artery. Optionally, one or more features of device 100 in the right branch artery 10 may be different than that of device 100 in left branch artery 10. It is noted that anchoring element 120 as well as other features of device 100 are not shown here for clarity purposes.

Structural features and details on materials sharing a same reference number can be used with any one of embodiments shown in figures (FIGS. 1A-8B). For example, details related to inner frame 135 discussed in reference to FIG. 2A can also be used in embodiments described in reference to FIGS. 3-8B.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A device comprising:
a constricting portion extending from an inlet at a proximal end for receiving blood flow therethrough to at least one outlet at a distal end through which flow is expelled; and
an anchor configured to anchor the device to an inner wall of a blood vessel;
wherein said constricting portion includes a neck section and wherein said constricting portion tapers from said inlet to said neck section and wherein said constricting portion comprises a flared section that flares from said neck section to said at least one outlet, having a distal end with a diameter larger than the diameter of said neck section and smaller than the diameter of said inlet and wherein said tapering and said flared section define a space substantially surrounding said constricting portion in which flow expelled from said at least one outlet can freely flow, wherein the device is configured to direct the bloodflow therethrough from said proximal end to said distal end, while the space allows backflow therewithin.

2. (canceled)

3. The device of claim 1, wherein said anchor is an outer frame defining an outer cylindrical wall with openings through which blood may freely flow.

4. The device of claim 3, further comprising an inner frame defining an inner cylindrical wall with openings through which blood may freely flow and a cover partially lining said inner frame, wherein said constriction portion is formed by a first portion of said inner frame that is lined with said cover, said first portion extending from said inlet to said at least one outlet.

5. The device of claim 4, wherein said inner frame is within said outer frame and oriented in a same direction.

6. The device of claim 5, wherein said inner frame and said outer frame extend from said proximal end to a frame distal end.

7-8. (canceled)

9. The device of claim 5, wherein said cover lines 50%-90% of a length of said inner frame.

10. (canceled)

11. The device of claim 5, wherein said cover is a liquid impermeable cover.

12. (canceled)

13. The device of claim 11, wherein a diameter of said neck section is 5 mm-15 mm.

14. The device of claim 11, wherein said anchor is fixed to said constricting portion at said proximal end and further comprises a circumferential sealing strip extending around said anchor at said proximal end.

15. The device of claim 14, wherein said circumferential sealing strip lines said anchor over a length of 3 mm-10 mm from said proximal end.

16. The device of claim 14, wherein said constricting portion is lined with a cover and wherein said circumferential sealing strip is connected to said cover of said constricting portion with a liquid impermeable sealed connection.

17. The device of claim 1, comprising a constrictor fitted around said neck section and configured to constrict said neck section to a defined diameter, and wherein said constrictor is configured to maintain said defined diameter while implanted within blood vessel.

18-19. (canceled)

20. A device comprising:
an outer frame defining an outer cylindrical wall with openings through which blood may freely flow and configured for being anchored within a blood vessel;
an inner frame defining an inner cylindrical wall with openings through which blood may freely flow, wherein said inner frame is within said outer frame, and wherein at least a portion of said inner frame is fixedly attached to said outer frame, said inner frame having an inner frame length that extends from a proximal end to a frame distal end;
a cover lining said inner frame from said proximal end to a cover distal end to form a constricting portion, wherein said constricting portion has a length that is shorter than said inner frame length, wherein said cover seals said openings in said inner frame over said length of said constricting portion;
a constrictor configured to constrict a diameter of said inner frame along said constricting portion to form a neck section with a defined neck diameter,
wherein said constricting portion tapers from said proximal end to said neck section and wherein said tapering defines a space substantially surrounding said constricting portion in which flow expelled from said constricting portion can freely flow.

21. The device of claim 20, wherein said cover lines 50%-90% of a length of said inner frame.

22. (canceled)

23. The device of claim 20, wherein a second portion of said inner frame extending from said cover distal end to said frame distal end is not lined with said cover and is exposed.

24. The device of claim 23, wherein an edge of said inner frame at said cover distal end is configured to be spaced away from an inner wall of a blood vessel when the device is implanted therein.

25. The device of claim 24, wherein a diameter of said inner frame at said cover distal end is at least 10% smaller than a diameter of said inner frame at said distal end.

26. The device of claim 20, wherein said outer frame is fixed to said inner frame at said proximal end and further comprising a circumferential sealing strip extending around said outer frame at said proximal end.

27. The device of claim 26, wherein said circumferential sealing strip lines said outer frame over a length of 3 mm-10 mm from said proximal end.

28. The device of claim 20, wherein a diameter of said neck section is 5 mm-15 mm.

29-30. (canceled)

31. The device of claim 1, wherein said space is configured to provide free blood flow into branch blood vessels along a length of the blood vessel in which the device is implanted.

32. The device of claim 1, wherein the device is configured for being implanted within a pulmonary artery.

33. The device of claim 32, wherein the device is configured for being implanted in the right or left pulmonary artery.

34. The device of claim 33, wherein the device is configured for elevating pressure in the right ventricle.

35. The device of claim 11, wherein said cover extends from said proximal end to said distal end, wherein said distal end is at a distance downstream said neck section, defining a flared section.

36. The device of claim 20, wherein said constricting portion comprises a flared section that flares from said neck section to said cover distal end, having a distal end with a diameter larger than the diameter of said neck section and smaller than the diameter of said proximal end.
